(19) United States
(12) Patent Application Publication
(10) Pub. No.: US 2026/0131289 A1
(43) Pub. Date: May 14, 2026

(54) Anion Exchange Separation Article, Methods of Making, and Methods of Using

(71) Applicant: Solventum Intellectual Properties Company, St Paul, MN (US)

(72) Inventors: Jerald Kenneth Rasmussen, St Paul, MN (US); George W. Griesgraber, St Paul, MN (US)

(73) Assignee: Solventum Intellectual Properties Company, St Paul, MN (US)

(21) Appl. No.: 19/118,758

(22) PCT Filed: Sep. 5, 2023
(86) PCT No.: PCT/IB2023/058780

§ 371 Date: Apr. 4, 2025

Related U.S. Application Data
(60) Provisional application No. 63/378,402, filed on Oct. 5, 2022.

(51) Int. Cl. B01D 67/00; B01D 69/02; B01D 71/40; B01J 41/04; B01J 41/14; B01J 47/12; C07C 279/12; C07C 279/14; C08J 5/22

(52) U.S. Cl. CPC B01D 67/00931; B01D 69/02; B01D 71/40; B01J 41/04; B01J 41/14; B01J 47/12; C07C 279/12; C07C 279/14; C08J 5/2275; B01D 2325/42; C08J 2377/06; C08J 2433/14

(57) ABSTRACT

An anion exchange separation article is provided that is useful for separation of complex samples that contain a mixture of materials having different ionic charges. The separation articles include a plurality of polymeric chains grafted to a porous polymeric substrate that is a solid. The plurality of polymeric chains extends away from a surface of the porous polymeric substrate and the side chains of the monomeric repeat units of the polymer chains are terminated with a guanidinium group or a salt thereof. The separation articles can be used, for example, for separation of biomaterials in a sample based on differences in their charges. Advantageously, the binding capacity of the anion separation articles is salt tolerant.

### BACKGROUND

Detection, quantification, isolation, and purification of target biomaterials, such as viruses and biomacromolecules (e.g., constituents or products of living cells such as proteins, carbohydrates, lipids, and nucleic acids) have long been objectives of investigators. Detection and quantification are important diagnostically, for example, as indicators of various physiological conditions such as diseases. Isolation and purification of biomacromolecules are important for therapeutic uses and in biomedical research.

Polymeric materials have been widely used for the separation and purification of various target biomaterials. Such separation and purification methods can be based on any of a variety of binding factors or mechanisms including the presence of an ionic group, the size of the target biomaterial, a hydrophobic interaction, an affinity interaction, the formation of a covalent bond, and so forth.

Membrane-based technologies, especially in a disposable format, are becoming increasingly important in biopharmaceutical and vaccine manufacturing processes. Membranes have been used in passive, size-based separations (for example, in virus removal applications) and, more recently, in active filtration (for example, for the removal of minor contaminants in later stages of purification processes).

Functionalized membranes (e.g., functional polymer-bearing membranes) have typically suffered from relatively low biomaterial binding capacities and this has generally limited their use in large-scale purifications. Additionally, many of these functionalized membranes have decreased binding capacity for biomaterials as the ionic strength of the samples increase. Therefore, porous chromatography resins bearing ion exchange or other interactive ligand functional groups have been typically used rather than functionalized membranes in “capture-and-elute” type purification processes such as for protein purification.

### SUMMARY

There is a need for new anion exchange separation articles that have a high binding capacity to various biomaterial such as proteins as well as good salt tolerance. That is, the binding capacity does not decrease dramatically with an increase in the ionic strength of the eluent and/or samples used during the separation process.

In a first aspect, an anion exchange separation article is provided that includes (1) a porous polymeric substrate that is a solid and (2) a plurality of polymeric chains grafted to the porous polymeric substate and extending away from a surface of the porous polymeric substate. The polymeric chains comprise monomeric units derived from a monomer of Formula (I)

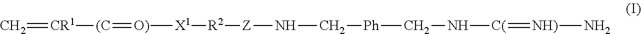

or a salt thereof. In Formula (I), group R¹ is hydrogen or methyl; X¹ is —O— or —NH—; R² is alkylene or heteroalkylene; Z is —NH—(C═O)— or —(C═O)—; and Ph is phenylene.

In a second aspect, a method of making an anion exchange separation article is provided. The method includes providing a porous polymeric substrate that is a solid and grafting a plurality of polymeric chains to the porous polymeric substrate. The polymeric chains comprise monomeric units derived from a monomer of Formula (I) as described above in the first aspect.

In a third aspect, a method of separating a mixture of materials is provided. The method includes providing an anion exchange separation article as described above in the first aspect and passing the mixture of materials through the anion exchange separation device, wherein the anion exchange separation device separates the mixture of materials based on their ionic charge.

In a fourth aspect, monomers of Formula (I) are provided as described in the first aspect.

### DETAILED DESCRIPTION

An anion exchange separation article is provided that is useful for separation of complex samples that contain a mixture of materials having different ionic charges. The separation articles include a plurality of polymeric chains grafted to a porous polymeric substrate that is a solid. The plurality of polymeric chains extends away from a surface of the porous polymeric substrate and contain a plurality of monomeric units having a guanidinium group or a salt thereof. The separation articles can be used, for example, for separation of biomaterials in a sample based on differences in their anionic charge or for separation of anionic (i.e., negatively charged) materials from cationic (i.e., positively charged) materials. Advantageously, the binding capacity of the anion exchange separation articles is salt tolerant.

Salt tolerance means that the binding capacity of the anion exchange separation articles typically does not decrease substantially when the ionic strength is increased. For example, most conventional anion exchange media lose 50 percent or more of its binding capacity if the ionic strength is increased from a low ionic strength of about 3 to 6 mM to a high ionic strength such as 50 millimolar (mM) or higher.

Salt tolerance of anion exchange media can be measured in comparison to that of the conventional quaternary ammonium ligand (e.g., trimethylammonium, or Q, ligand), whose primarily electrostatic interactions with biological species rapidly deteriorates at conductivities three- to six-fold less than the target range. For example, membranes functionalized with the conventional Q ligand exhibit a drop in φX174 viral clearance from a six (6) log reduction value (LRV) to a one (1) LRV in going from 1 to 50 mM NaCl (ca. 5-6 mS/cm conductivity). Viruses such as φX174 that have isoelectric points (pI's) close to 7 (are neutral or near neutral) are extremely difficult to remove from process streams. Similar problems are observed when attempting to remove other biological species from process fluids. For example, when trying to remove positively charged proteins such as host cell proteins using filtration devices functionalized with conventional Q ligands, the process fluid may have to be diluted two-fold or more to reduce the conductivity to an acceptable range. This is expensive and dramatically increases overall processing time.

Surprisingly, the anion exchange separation devices described herein can be effective in capturing target biological species even in the presence of high ionic strength conditions. For example, the salt (e.g., NaCl) concentration can be as high as 50, 75, 100, 125, 150, 175, 200, 225, 250, 275, or even 300 mM or higher. In some embodiments, the binding capacity remains constant or increases with ionic strength. In other embodiments, the binding capacity at 250 mM ionic strength may decrease no more than 10%, 20%, 35%, or 50% of that measured in low ionic strength media (e.g., 3 or 6 mM). Surprisingly, the salt tolerance of the anion exchange articles described herein is quite unusual. For example, the binding capacity for bovine serum albumin (BSA) can be maintained up to 250 mM ionic strength using the anion exchange articles described herein whereas an anion exchange article with trimethylammonium ligands loses over 40 percent of its capacity at 50 mM and 90 percent of its capacity at 250 mM ionic strength.

As used herein, the terms “a”, “an”, “the”, and “at least one” are used interchangeably.

The term “and/or” means either or both. For example, “A and/or B” means A alone, B alone, or both A and B.

The term “guanidinium” refers to a monovalent group of formula —NH—C(═NH)—NH₂. A salt of the guanidinium group is a cationic group that is charged balanced with a counter anion. Any suitable counter anion can be used such as, for example, halides, sulfates, phosphates, and the like.

The term “(hetero)alkylene” refers to an alkylene, heteroalkylene, or both.

The term “alkylene” refers to a divalent group that is a radical of an alkane. The alkylene group can have 1 to 32 carbon atoms, 1 to 20 carbon atoms, 1 to 12 carbon atoms, 1 to 10 carbon atoms, 1 to 6 carbon atoms, or 1 to 4 carbon atoms. The alkylene can be linear, branched, cyclic, or a combination thereof. A linear alkylene has at least one carbon atom while a cyclic or branched alkylene has at least 3 carbon atoms.

The term “heteroalkylene” refers to an alkylene in which one or more of the catenated carbon atoms is replaced with a heteroatom such as oxygen, nitrogen, or sulfur. There are not two heteroatoms adjacent to each other as in a peroxide. That is, if there is more than one heteroatom, the heteroatoms are separated from each other by at least one carbon atom.

The term “grafted” is used to indicate that polymeric chains are covalently attached to the porous polymeric substrate. In most embodiments, the polymeric chains are grafted to a carbon atom in the polymeric backbone of the porous polymeric substrate.

### Anion Exchange Separation Articles

The anionic exchange separation articles have a porous polymeric substrate that is a solid. The term “solid” in reference to the porous polymeric substrate means that the substrate is not a liquid and is not dissolved in a solution. The pores of the porous polymeric substrate can have any desired average size. In some embodiments, the pores are macro-porous, mesoporous, microporous, or a mixture thereof. As used herein, the term “macro-porous” refers to a polymeric substrate having pores with diameters greater than 50 nanometers, the term “meso-porous” refers a polymeric substrate having pores with diameters in a range of 2 nanometers to 50 nanometers, and the term “micro-porous” refers to a material having pores with diameters less than 2 nanometers.

The terms “solid porous polymeric substrate”, “porous polymeric substrate”, “polymeric substrate”, “substrate”, and similar variations can be used interchangeably herein.

The porous polymeric substrate can have any desired size, shape, and form. For example, the porous polymeric substrate can be in the form of particles, fibers, films, non-woven webs, woven webs, membranes, sponges, or sheets. In some examples, the polymeric substrate is a porous membrane or a porous non-woven web. To prepare large separation articles or many separation articles and for ease of manufacturing, the polymeric substrate can be in the form of or formed from a roll such as a roll of film, non-woven web, woven web, membrane, sponge, or sheet. This allows the use of roll-to-roll processing to prepare the separation articles. The porous polymeric substrate can include a single layer or multiple layers of the same or different polymeric materials.

The porous polymeric substrate is often formed from a thermoplastic material. Suitable thermoplastics include, but are not limited to, polyolefins, poly(isoprenes), poly(butadienes), fluorinated polymers, chlorinated polymers, polyamides, polyimides, polyethers, poly(ether sulfones), poly(sulfones), poly(vinyl acetates) and copolymers thereof such as poly(ethylene)-co-poly(vinyl acetate), polyesters such as poly(lactic acid), poly(vinyl alcohol) and copolymers thereof such as poly(ethylene)-co-poly(vinyl alcohol), poly(vinyl esters), poly(vinyl ethers), poly(carbonates), polyurethanes, poly((meth)acrylates) and copolymers thereof, and combinations thereof.

Suitable polyolefins for the porous polymeric substrate include poly(ethylene), poly(propylene), poly(1-butene), copolymers of ethylene and propylene, alpha olefin copolymers (such as copolymers of ethylene or propylene with 1-butene, 1-hexene, 1-octene, and/or 1-decene), poly(ethylene-co-1-butene), poly(ethylene-co-1-butene-co-1-hexene), poly(butadiene) and copolymers thereof, and combinations thereof.

Suitable fluorinated polymers for the porous polymeric substrate include poly(vinyl fluoride), poly(vinylidene fluoride), copolymers of vinylidene fluoride (such as poly(vinylidene fluoride-co-hexafluoropropylene)), copolymers of chlorotrifluoroethylene (such as poly(ethylene-co-chlorotrifluoroethylene)), and combinations thereof.

Suitable polyamides for the porous polymeric substrate include various nylon compositions such as, for example, poly(iminoadipoyliminohexamethylene), poly(iminoadipoyliminodecamethylene), polycaprolactam, and combinations thereof. Suitable polyimides include poly(pyromellitimide), and combinations thereof.

Suitable poly(ether sulfones) for the porous polymeric substrate include poly(diphenylether sulfone), poly(diphenylsulfone-co-diphenylene oxide sulfone), and combinations thereof.

Suitable copolymers of vinyl acetate for the porous polymeric substrate include copolymers of ethylene and vinyl acetate as well as terpolymers of vinyl acetate, vinyl alcohol, and ethylene.

In some embodiments, the porous polymeric substrate is a porous membrane having an average pore size (average longest diameter of the pore) that is often greater than 0.1 micrometer to minimize size exclusion separations, minimize diffusion constraints, and maximize surface area and separation. Generally, the average pore size can be in the range of 0.1 to 10 micrometers. For example, the average pore size is at least 0.2 micrometers, at least 0.4 micrometers, at least 0.6 micrometers, or at least 0.8 micrometers and up to 8 micrometers, up to 6 micrometers, up to 4 micrometers, or up to 2 micrometers.

The porous polymeric substrate can be a macro-porous membrane such as a thermally induced phase separation (TIPS) membrane. TIPS membranes are often prepared by forming a solution of a thermoplastic material and a second material above the melting point of the thermoplastic material. Upon cooling, the thermoplastic material crystallizes and phase separates from the second material. The crystallized material is often stretched. The second material is optionally removed either before or after stretching. Macro-porous membranes are further described in U.S. Pat. No. 4,539,256 (Shipman), U.S. Pat. No. 4,726,989 (Mrozinski), U.S. Pat. No. 4,867,881 (Kinzer), U.S. Pat. No. 5,120,594 (Mrozinski), U.S. Pat. No. 5,260,360 (Mrozinski), and U.S. Pat. No. 5,962,544 (Waller, Jr.). Some exemplary TIPS membranes include poly(vinylidene fluoride) (PVDF), polyolefins such as poly(ethylene) or poly(propylene), vinyl-containing polymers or copolymers such as ethylene-vinyl alcohol copolymers and butadiene-containing polymers or copolymers, and (meth)acrylate-containing polymers or copolymers. TIPS membranes including PVDF are further described in U.S. Pat. No. 7,338,692 (Smith et al.).

In some embodiments, the porous polymeric substrate can include a nylon macro-porous film or sheet (for example, a macro-porous membrane), such as those described in U.S. Pat. No. 6,056,529 (Meyering et al.), U.S. Pat. No. 6,267,916 (Meyering et al.), U.S. Pat. No. 6,413,070 (Meyering et al.), U.S. Pat. No. 6,776,940 (Meyering et al.), U.S. Pat. No. 3,876,738 (Marinaccio et al.), U.S. Pat. No. 3,928,517 (Knight et al.), U.S. Pat. No. 4,707,265 (Barnes, Jr. et al.), and U.S. Pat. No. 5,458,782 (Hou et al.).

In other embodiments, the porous polymeric substrate can be a nonwoven web, which can include nonwoven webs manufactured by any of the commonly known processes for producing nonwoven webs. As used herein, the term “nonwoven web” refers to a fabric that has a structure of individual fibers or filaments that are randomly and/or unidirectionally interlaid in a mat-like fashion.

For example, the fibrous nonwoven web can be made by wet laid, carded, air laid, spunlaced, spunbonding, or melt-blowing techniques, or combinations thereof. Spunbonded fibers are typically small diameter fibers that are formed by extruding molten thermoplastic polymer as filaments from a plurality of fine, usually circular capillaries of a spinneret, with the diameter of the extruded fibers being rapidly reduced. Melt-blown fibers are typically formed by extruding molten thermoplastic material through a plurality of fine, usually circular, die capillaries as molten threads or filaments into a high velocity, usually heated gas (for example, air) stream, which attenuates the filaments of molten thermoplastic material to reduce their diameter. Thereafter, the melt-blown fibers are carried by the high velocity gas stream and are deposited on a collecting surface to form a web of randomly dispersed, melt-blown fibers. Any of the nonwoven webs can be made from a single type of fiber or from two or more fibers that differ in the type of thermoplastic polymer and/or thickness.

Further details of manufacturing methods of useful nonwoven webs have been described by Wente in “Superfine Thermoplastic Fibers,” Indus. Eng. Chem., 48, 1342 (1956) and by Wente et al. in “Manufacture of Superfine Organic Fibers,” Naval Research Laboratories Report No. 4364 (1954).

The nonwoven web substrate may optionally further comprise one or more layers of scrim. For example, cither or both major surfaces of the nonwoven web may each optionally further comprise a scrim layer. The scrim, which is typically a woven or nonwoven reinforcement layer made from fibers, is included to provide strength to the nonwoven web. Suitable scrim materials include, but are not limited to, nylon, polyester, fiberglass, polyethylene, polypropylene, and the like. The average thickness of the scrim can vary but often ranges from about 25 to about 100 micrometers, preferably about 25 to about 50 micrometers. The scrim layer may optionally be bonded to the nonwoven article. A variety of adhesive materials can be used to bond the scrim to the nonwoven. Alternatively, the scrim may be heat-bonded to the nonwoven web.

The porosity of nonwoven substrates is typically characterized by properties such as fiber diameter, or basis weight, or solidity, rather than by pore size. The fibers of the nonwoven substrate are typically microfibers having an effective fiber diameter of at least 0.5, 1, 2, or even 4 micrometers and at most 15, 10, 8, or even 6 micrometers, as calculated according to the method set forth in Davies, C. N., “The Separation of Airborne Dust and Particles,” Institution of Mechanical Engineers, London, Proceedings 1B, 1952. The nonwoven substrate preferably has a basis weight in the range of at least 5, 10, 20, or even 50 g/m²; and at most 800, 600, 400, 200, or even 100 g/m². The minimum tensile strength of the nonwoven web is about 4.0 Newtons. It is generally recognized that the tensile strength of nonwoven substrates is lower in the machine direction than in the cross-web direction due to better fiber bonding and entanglement in the latter.

Nonwoven web loft is measured by solidity, a parameter that defines the solids fraction in a volume of web. Lower solidity values are indicative of greater web loft. Solidity (α) is a unitless fraction typically represented by: α=m_{f}÷ρ_{f}×L_{nonwoven} where m_{f} is the fiber mass per sample surface area, ρ_{f} is the fiber density, and L_{nonwoven} is the nonwoven thickness. Solidity is used herein to refer to the nonwoven substrate itself and not to the functionalized nonwoven substrate. When a nonwoven substrate contains mixtures of two or more kinds of fibers, the individual solidities are determined for each kind of fiber using the same L_{nonwoven} and these individual solidities are added together to obtain the web's solidity, α.

The polymeric chains grafted to the porous polymeric substrate comprise monomeric units derived from a monomer of Formula (I).

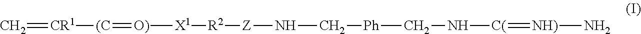

or a salt thereof. In Formula (I), group R¹ is hydrogen or methyl; X¹ is —O— or —NH—; R² is a (hetero)alkylene; Z is —NH—(C═O)— or —(C═O)—; and Ph is phenylene. The monomers are often grafted to a carbon atom in a polymeric backbone of the polymeric material included in the porous polymeric substrate. That is, the grafted polymeric chain, if a homopolymer, is of formula

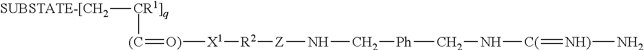

with the attachment to a carbon atom in the substrate. The variable q is the number of monomeric units in the polymeric chain.

The group R² can be an alkylene or a heteroalkylene. Suitable alkylenes typically have 1 to 20 carbon atoms such as at least 1, at least 2, at least 3, at least 4, or at least 5 and up to 20, up to 18, up to 14, up to 12, up to 10, up to 8, or up to 6 carbon atoms. Suitable heteroalkylene typically have 2 to 20 carbon atoms and 1 to 5 heteroatoms. The number of carbon atoms in the heteroalkylene can be at least 2, at least 3, at least 5, at least 6 and up to 20, up to 18, up to 14, up to 12, up to 10, up to 8, or up to 6 carbon atoms. The heteroatoms can be oxygen (e.g., the group-O—) or nitrogen (e.g., the group-NH—). In many embodiments the heteroatoms are oxygen. Some specific R² groups include, but are not limited to, —C(CH₃)₂—, —(CH₂C(CH₃)₂—, —CH₂CH₂—, —CH₂CH₂CH₂—, and —CH₂CH₂—(O—CH₂CH₂)ₓ— where x is an integer in a range of 1 to 5 or 1 to 3.

The two methylene groups attached to the phenylene (—CH₂-Ph-CH₂—) in Formula (I) can be in either a meta or para configuration.

The monomers of Formula (I) can be prepared, for example, by initially reacting a xylenediamine (1) with O-methylisourea hemisulfate (2) as shown in Reaction Scheme A. For ease of explanation, the compound of formula (2) is shown without the hemisulfate counterion.

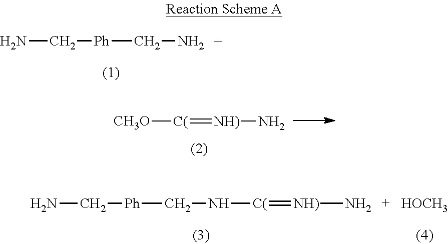

The reaction product is a compound of formula (3). This intermediate compound can be reacted with an isocyanato-containing monomer as shown in Reaction Scheme B or with an alkenylazlactone compound as shown in Reaction Scheme C.

Reaction Scheme B is shown below for reacting with an isocyanato-containing monomer of compound (5) with compound (3) of Reaction Scheme A to form compound (6).

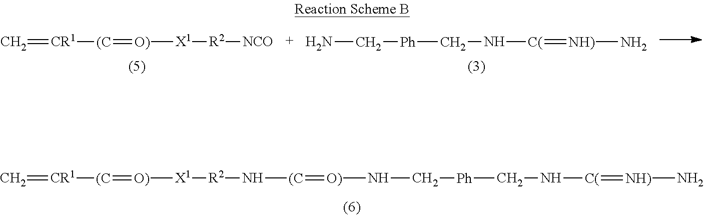

The groups R¹, X², and R² in the isocyanato-containing compound (5) are the same as described above. In many embodiments using Reaction Scheme B, R¹ is methyl and R² is an alkylene such as ethylene or propylene. Compound (6) corresponds to the compound of Formula (I) where Z is equal to —NH—(C═O)—.

In an alternative method of preparing a compound of Formula (I), compound (3) of Reaction Scheme A is reacted with an alkenyl azlactone compound. The alkenyl azlactone compound is often compound (7) shown in Reaction Scheme C.

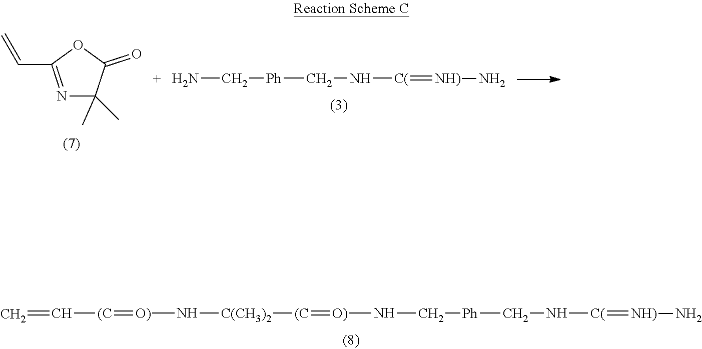

Compound (8) corresponds to Formula (I) where R¹ is hydrogen, X1 is —NH—, R² is —C(CH₃)₂—, and Z is —(C═O)—.

Example monomers of Formula (I) include, but are not limited to, the following compounds or salts thereof where R¹ is hydrogen or methyl.

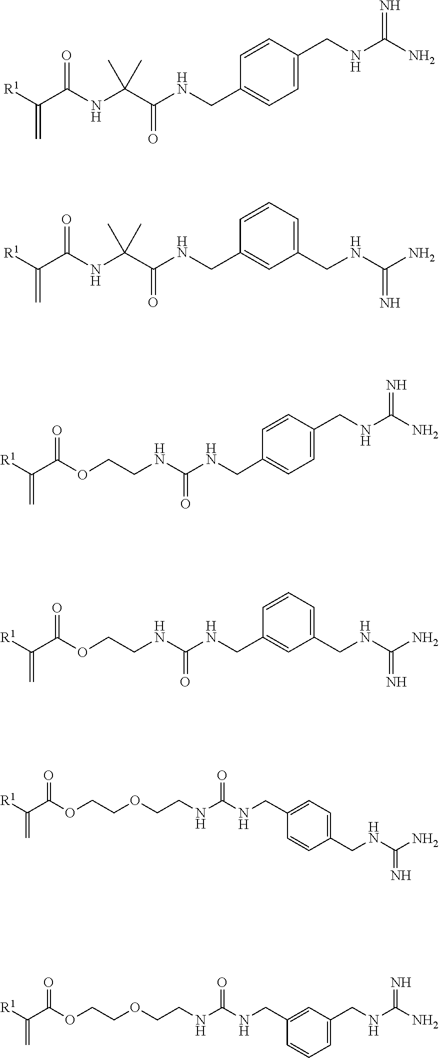

The polymeric chains grafted to the porous polymeric substrate can be a homopolymer or co-polymer. The polymeric chains are often homopolymers of the monomers of Formula (I) to prepare polymers with high binding capacity for the materials desired to be captured. That is, the polymeric chains can contain up to 100 weight percent of first monomers of Formula (I) based on the total weight of monomers used to form the polymeric chains. In some embodiments, other monomers (second monomers) can be copolymerized with the first monomers to adjust the binding capacity and/or to achieve other desired properties of the polymeric chains. Any suitable second monomer can be used but they are typically hydrophilic monomers. For example, they are often water soluble or water miscible.

The amount of the first monomer of Formula (I) can be, for example, in a range of 20 to 100 weight percent based on the total weight of monomeric units in the polymeric chain. The amount can be at least 20, at least 30, at least 40, at least 50, at least 60, at least 70, at least 75, at least 80, at least 85, at least 90, or at least 95 and up to 100, up to 99, up to 98, up to 97, up to 95, up to 90, up to 85, up to 80, or up to 75 weight percent based on the total weight of monomeric units in the polymeric chain. Higher amounts of the first monomer tend to increase the binding capacity for various target compounds such as biomaterials. In many embodiments, the amount of the first monomer of Formula (I) is in a range of 80 to 100, 85 to 100, 90 to 100, or 95 to 100 weight percent based on the total weight of monomeric units.

The optional second monomer in the polymeric chain can be, for example, a hydrophilic monomer to adjust the degree of hydrophilicity imparted to the substrate. The hydrophilic monomer has an ethylenically unsaturated group and a hydrophilic group such as, for example, a hydroxyl group, ether group, or amido group. Suitable hydrophilic monomers include acrylamide, dimethylacrylamide, hydroxyethyl(meth)acrylate, hydroxypropyl(meth)acrylate, ethoxyethylmethacrylate, diethyleneglycolmethylether methacrylate, 2-hydroxyethylacrylamide, N-vinylpyrrolidone, and the like, and combinations thereof.

Other optional second monomers include those that have more than one ethylenically unsaturated group. This types of second monomers are typically water soluble and are used in only relatively small amounts to impart a degree of branching and/or relatively light crosslinking to a resulting copolymer. For example, the amount of these multifunctional monomers having more than two ethylenically unsaturated groups may be present in an amount ranging from 0.1 to 25 weight percent, based upon the total weight of monomers in the first polymerizable composition. The amount can be at least 0.1, at least 0.2, at least 0.5, or at least 1.0 weight percent and up to 25, up to 20, up to 15, up to 10, up to 5, up to 4, up to 3, up to 2, or up to 1 weight percent. Although crosslinking monomers can be used and can be beneficial for some applications, they tend to reduce binding capacity for some biomaterials.

Examples include, but are not limited to, poly(ethyleneglycoldi(meth)acrylate, methylenebisacrylamide, 3-acryloyloxy-2-hydroxypropyl methacrylate, glyceroldimethacrylate, glyceroldiacrylate, diacryloylpiperazine, and 1,2-ethylenebisacrylamide.

The total amount of the second monomer can be up to 80 weight percent of the monomers used to form the polymeric chain. Lower amounts of the second monomer typically enhance the binding capacity for various target compounds such as protein biomaterials. The amount, if present, is usually equal to 100 minus the weight percent of the first monomer of Formula (I) based on the total weight of monomers in the first polymerizable composition.

The polymeric chains are grafted onto the porous polymeric substrate. Any suitable method of grafting can be used. In many embodiments, a Type II photoinitiator is combined with the monomer composition to form a reaction mixture. Upon exposure of the reaction mixture to ultraviolet radiation, the Type II photoinitiator abstracts a hydrogen atom from the porous polymeric substrate resulting in the generation of free radicals on the porous polymeric substrate. The free radicals react with the monomers present in the composition resulting in the formation of polymeric chains grafted to the porous polymeric substrate. The polymeric chains are often grafted to a carbon atom in the backbone of the polymeric material contained in the porous polymeric substrate.

Type II photoinitiators are typically aromatic ketone compounds. Examples include, but are not limited to, benzophenone, carboxybenzophenone (e.g., 3-carboxy benzophenone), 4-(3-sulfopropyloxy)benzophenone sodium salt, Michler's ketone, benzil, anthraquinone, 5,12-naphthacenequinone, accanthracenequinone, benz(A)anthracene-7,12-dione, 1,4-chrysenequinone, 6,13-pentacenequinone, 5,7,12,14-pentacenetetrone, 9-fluorenone, anthrone, xanthone, thioxanthone, 2-(3-sulfopropyloxy)thioxanthen-9-one, acridone, dibenzosuberone, acetophenone, and chromone.

The ultraviolet (UV) light used to generate free radicals on the porous polymeric substrate can be provided by various light sources such as light emitting diodes (LEDs), black lights, medium pressure mercury lamps, etc., or a combination thereof. The actinic radiation (e.g., UV radiation) can also be provided using higher intensity light sources such as those available from Fusion UV Systems Inc. The ultraviolet light sources can be relatively low light intensity sources such as blacklights that provide generally 10 mW/cm² or less (as measured in accordance with procedures approved by the United States National Institute of Standards and Technology as, for example, with a UVIMAP™ UM 365 L-S radiometer manufactured by Electronic Instrumentation & Technology, Inc., in Sterling, VA) over a wavelength range of 280 to 400 nanometers. Alternatively, relatively high light intensity sources such as medium pressure mercury lamps can be used that provide intensities generally greater than 10 mW/cm², preferably between 15 and 450 mW/cm². The exposure time can be up to about 30 minutes or even longer.

In another method useful for generating free radicals on a surface of the porous polymeric substrate, the substrate itself is selected to be photoactive and no Type II photoinitiator is needed. The monomer composition is exposed to actinic radiation, which is typically in the ultraviolet region of the electromagnetic spectrum. Upon exposure to the actinic radiation, the polymeric substrate absorbs enough energy that some of its covalent bonds are broken, resulting in the generation of free radicals that can react with the monomers to form polymeric chains. Examples of photoactive polymeric substrates include polysulfones and poly(ether sulfones). Other photoactive polymeric substrates often contain an aromatic group such as, for example, homopolymers and block copolymers of poly(methylphenylsilane) and various polyimides based on benzophenone tetracarboxylic dianhydride.

In other methods for generating free radicals on a surface of the polymeric substrate, ionizing radiation is used rather than a Type II photoinitiator and/or UV radiation. As used herein, the term “ionizing radiation” refers to radiation that is of a sufficient dose and energy to form free radical reaction sites on the surface and/or in the bulk of the polymeric substrate. The radiation is of sufficient energy if it is absorbed by the polymeric substrate and results in the cleavage of chemical bonds in the substrate and the formation of free radicals. The ionizing radiation is often beta radiation, gamma radiation, electron beam radiation, x-ray radiation, plasma radiation, or other suitable types of electromagnetic radiation. Preferably, ionizing radiation is conducted in an inert environment to prevent oxygen from reacting with the radicals.

In many embodiments of this process, the ionizing radiation is electron beam radiation, gamma ray radiation, x-ray radiation, or plasma radiation because of the ready availability of suitable generators. Electron beam generators are commercially available such as, for example, the ESI ELECTROCURE EB SYSTEM from Energy Sciences, Inc. (Wilmington, MA, USA) and the BROADBEAM EB PROCESSOR from E-beam Technologies (Davenport, IA, USA). Gamma ray radiation generators are commercially available from MDS Nordion that use a cobalt-60 high energy source.

For any given type of ionizing radiation, the dose delivered can be measured in accordance with ISO/ASTM52628-13, “Standard Practice for Dosimetry in Radiation Processing,” by ASTM International (West Conshohocken, PA). By altering the extractor grid voltage, beam diameter, exposure time, and distance from the irradiation source, various dose rates can be obtained.

Multiple polymeric chains are grafted to the porous polymeric substrate. The term “ligand density” refers to the millimoles of monomeric units per gram grafted to a substrate. The millimoles are calculated by dividing the mass gain by the molecular weight of the monomer and multiplying by 1000. This value is then normalized by dividing by the original mass (grams) of the porous polymeric substrate. The ligand density (mmoles/gram) is expressed as millimoles of monomeric units grafted per gram of substrate. For clarity, the material that is grafted is typically a polymeric material containing a plurality of monomeric units.

When the substrate is a membrane, the anion exchange separation articles often have a ligand density of about 0.02 to about 3 mmoles/gram or even higher. The graft density can be at least 0.02, at least 0.05, at least 0.1, at least 0.2, at least 0.5, or at least 1 mmoles/gram and up to 3, up to 2.5, up to 2, up to 1.5, up to 1, up to 0.8, up to 0.7, or up to 0.5 mmoles/gram. The weight gain is calculated from the equation [100 (Weight 2−Weight 1)÷Weight 1] where Weight 1 is the weight of the substrate and Weight 2 is the weight of the substrate with grafted polymers attached. The weight gain can be in a range of 1 to 85 weight percent or even higher. The amount, can be, for example, at least 1, at least 2, at least 5, at least 10, at least 15, at least 20, at least 25, at least 30, at least 35, at least 40, at least 45, or at least 50 weight percent and up to 85, up to 80, up to 75, up to 70, up to 65, up to 60, up to 55, up to 50, up to 45, up to 40, up to 35, or up to 30 weight percent.

When the substrate is a nonwoven or fibrous substrate, the weight gain upon grafting can often be higher than that of membrane substrates. The weight gain can be in a range of 20 to 400 weight percent or even higher. The amount can be, for example, at least 20, at least 50, at least 100, at least 150, at least 200, at least 250, or at least 300 weight percent and up to 400, up to 350, up to 300, up to 250, up to 200, up to 150, up to 100, up to 75, or up to 50 weight percent. For example, the weight gain can be in a range of 100 to 400, 100 to 300, or 100 to 200 weight percent.

The efficiency of binding (i.e., ligand efficiency) can be calculated by dividing the moles of ligands by the moles of biomaterial (e.g., protein) sorbed. The lower this number, the more effective is the anion separation article for sorbing biomaterials. This number is often dependent on the size of the biomaterial. For example, the ligand efficiency for small biomaterials can be about 10 or more while the ligand efficiency for large biomaterials can up to 1000 or even greater. Surprisingly, anion exchange separation devices prepared, for example, from the VDM adduct of 1-(4-(aminomethyl)benzyl)guanidine sulfate had a binding efficiency for bovine serum albumin (BSA) of more than double that of anion exchange separation devices prepared from the IEM adduct of agmatine, which is a compound not of Formula (I). This data is included in Tables 1 and 2 of the Example Section.

The anion exchange separation article is salt tolerant which means that it can be used under conditions of high ionic strength. As used herein in reference to salt tolerance, the term “salt” includes all low molecular weight ionic species that contribute to the conductivity of the solution. Salt tolerance is important because many of the aqueous processing solutions used in biopharmaceutical or enzyme manufacturing processes have conductivities in the range of 15-30 mS/cm (approximately 150-300 mM ionic strength) or more.

In typical ion exchange (IEX) processes, the binding of proteins or other biological species to the ionic ligands of the IEX support decreases as the ionic strength (salt concentration) increases. This is due to electrostatic screening by the salt ions in solution. Such processes are not salt tolerant.

Protein-based drugs, including monoclonal antibodies (mAbs) are typically purified by a series of chromatography steps. Often, two or more of these steps are IEX chromatography steps. Typical IEX chromatography media require low ionic strength buffer solutions for the proteins to interact with the IEX ligands. As a result, the protein solution collected from a previous chromatography step must often be diluted to lower the salt concentration before loading onto an IEX medium. This can be very expensive (high buffer and purified water cost), potentially requiring larger or additional holding tanks to accommodate the larger volumes of solution, and can be very time consuming, leading to overall increased manufacturing costs. Thus, the development of “salt-tolerant” ligands, that is, ligands that allow loading of protein solutions at relatively high ionic strength, obviating the need for dilution, is very important.

### EXAMPLES

### Materials and Methods

2-Vinyl-4,4-dimethylazlactone (VDM) was obtained from SNPE, Inc., and redistilled before use.

2-Isocyanatoethylmethacrylate (IEM) and 2-(2-isocyanatoethoxy)ethyl methacrylate (Karenz MOI-EG, CAS. No. 107023-60-9) were from Showa Denko KK, Tokyo, Japan. Methacrylamidopropyltrimethylammonium chloride (MAPTAC) and 3-(N-morpholino) propanesulfonic acid (MOPS) were obtained from the Sigma-Aldrich Company, St. Louis, MO.

3-Carboxybenzophenone was obtained from Sigma-Aldrich. A solution of 3-carboxybenzophenone, sodium salt (C-BP) (0.033 g/mL) was prepared by dissolving 3-carboxybenzophenone in IM sodium hydroxide and diluting with deionized water.

4-Aminobenzylamine and p-xylenediamine were obtained from TCI America, Portland, OR.

O-Methylisourea hydrochloride, O-methylisourea hemisulfate, and m-xylenediamine were obtained from Thermo Fisher Scientific, Waltham, MA.

TRIS (tris(hydroxymethyl)aminomethane) was obtained from JT Baker, Phillipsburg, NJ.

### Preparation of 1-(4-(aminomethyl)benzyl)guanidine Sulfate

A stirred solution of p-xylenediamine (50.0 g, 368 mmol) dissolved in 150 mL of methanol was cooled in an ice bath and treated with O-methylisourea hemisulfate (12.9 g, 105 mmol) followed by dropwise addition of concentrated sulfuric acid (5.14 g, 52.4 mmol). A white precipitate formed. The ice bath was removed and stirring was continued overnight. The resulting white solid was isolated by filtration and rinsed with several small portions of methanol. The white solid was treated with 200 ml of water and the mixture was heated to reflux. The mixture was then stirred for an additional 15 minutes. The stirred mixture was slowly cooled and then placed in an ice bath for 15 minutes. The resulting solid was isolated by filtration, rinsed with water, and air dried to provide 23.2 g of 1-(4-(aminomethyl)benzyl)guanidine sulfate as a white solid. ¹H-NMR (500 MHz, D₂O with a drop of NaOD in D₂O) d 7.15 (br s, 4H), 4.09 (s, 2H), 3.56 (s, 2H).

### Preparation of 1-(3-(aminomethyl)benzyl)guanidine Sulfate

A stirred solution of m-xylenediamine (50.0 g, 368 mmol) dissolved in 150 mL of methanol was cooled in an ice bath and treated with O-methylisourea hemisulfate (12.9 g, 105 mmol) followed by dropwise addition of concentrated sulfuric acid (5.14 g, 52.4 mmol). A white precipitate formed. The ice bath was removed and stirring was continued overnight. The resulting white solid was isolated by filtration and rinsed with several small portions of methanol. The white solid was crystallized (water/methanol) to provide 24.0 g of 1-(3-(aminomethyl)benzyl)guanidine sulfate as white crystals. ¹H-NMR (500 MHz, D₂O with a drop of NaOD in D₂O) d 7.35 (m, 1H), 7.30-7.25 (m, 3H), 4.32 (s, 2H), 4.05 (s, 2H).

### General Procedure for Membrane Coating and UV Irradiation Grafting

Coating solutions were prepared by mixing monomer solutions as prepared with deionized water and C-BP photoinitiator (varying amounts of a 0.033 g/mL solution in deionized water) to provide a mixture of the desired monomer and photoinitiator concentrations. Weight % solids of the prepared monomer solutions were measured and used to calculate dilution protocols for each coating experiment. A 9 cm×12 cm section of nylon membrane substrate (nylon 6,6 membrane, single reinforced layer nylon three-zone membrane, nominal pore size 0.8 microns, #080ZN, obtained from the 3M Company, St. Paul, MN) was placed on a sheet of polyester film and approximately 4.5 mL of a coating solution was pipetted onto the exposed surface of the membrane. The solution was allowed to soak into the membrane for about 1 minute and then a second polyester film was placed on top of the substrate. A 2.28 Kg cylindrical weight was rolled over the 3-layer sandwich to squeeze out any excess coating solution. UV irradiation grafting was conducted using a UV light stand (Classic Manufacturing, Inc., Oakdale, MN) equipped with 18 bulbs (Sylvania RG2 40W F40/350BL/ECO, 10 bulbs positioned above the membrane and 8 bulbs positioned below the membrane, 46 inches long, spaced 2 inches on center), with an irradiation time of 15 minutes. The polyester sheets were removed, and the polymer grafted membrane was placed in a 250 mL polyethylene bottle. The bottle was filled with 0.9 weight % saline (NaCl) solution, sealed, and shaken for 30 minutes to wash any residual monomer or ungrafted polymer from the membrane. In a second membrane wash step, the saline solution was decanted, the bottle was filled with deionized water, sealed, and then shaken for 30 minutes. The wash procedure was repeated three additional times, washing once using 0.9% saline solution followed by washing two times with deionized water. The grafted membrane was removed from the bottle and allowed to air dry. Each grafted membrane was analyzed for polymer graft density and static BSA binding capacity, from which ligand efficiencies were calculated.

### Static (Equilibrium) BSA Binding Capacity Method for Functionalized Membranes

Individual disks (16 mm diameter) of polymer grafted membranes were die-punched from sheets of polymer grafted membranes. A single disk was placed in a 5 mL centrifuge tube that contained 4.5 mL of bovine serum albumin (BSA, Sigma-Aldrich) prepared at a concentration of about 4 mg/mL in 25 mM TRIS buffer (pH 8.0, 50 mM NaCl). Each centrifuge tube was capped and tumbled overnight (typically 14 hours) on a rotating mixer. The resulting supernatant solution was analyzed using a UV-VIS spectrometer at 280 nm (with background correction applied at 325 nm). The static binding capacity for each disk was determined by comparison to the absorption value of the starting BSA solution, and results are reported in mg/mL (i.e., mg of BSA bound to membrane/mL of membrane volume) and reported as the average of three replicates.

### Determination of Ligand Density and Ligand Efficiency (Molar Ratio)

Ligand Density was determined based on mass gained by the membrane sample after the grafting procedure. First, the number of millimoles of ligand monomer grafted to each membrane sample was calculated by dividing the mass gain of the membrane sample by the molecular weight of the grafting monomer. The Ligand Density was then calculated by dividing the millimoles of ligand monomer grafted to membrane sample by the original mass of the membrane sample (expressed as millimoles of ligand monomer grafted per gram of membrane substrate (mmol/g)).

The Ligand Efficiency was determined by first converting the calculated Ligand Density to a volumetric basis using the measured bulk density of the membrane (0.415 g/mL) and then converting the calculated BSA Binding Capacity to a molar basis using the BSA molecular weight. The reported Ligand Efficiency (molar ratio of ligands per BSA molecule) was expressed as the quotient of Ligand Density to BSA Binding Capacity.

### Salt Tolerance Test Method for BSA Binding Capacity

Aqueous buffer solutions of 0.01 M MOPS (pH 7.0) were prepared. The ionic strengths (IS) of the buffer solutions were adjusted by the addition of varying amounts of sodium chloride. BSA solutions at about 3 mg/mL were prepared using the buffer solutions to provide individual BSA protein challenge solutions at 6, 50, 150, and 250 mM ionic strength. Membrane samples were tested with the challenge solutions according the “Static (Equilibrium) BSA Binding Capacity Method for Functionalized Membranes” described above.

### Example 1. Adduct of VDM and 1-(4-(aminomethyl)benzyl)guanidine

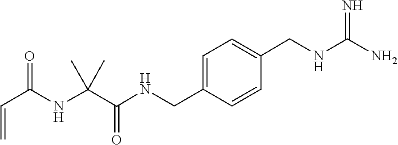 [SMILES: C=CC(=O)NC(C)(C)C(=O)NCc1ccc(CNC(=N)N)cc1]

1-(4-(aminomethyl)benzyl)guanidine sulfate (20.4 g, 74 mmol) was dissolved in IN NaOH (74 mL) with gentle heating and stirring. VDM (10.3 g, 74 mmol) was added dropwise to the stirred solution over a period of 4 minutes. After stirring for 6 hours, ¹H-NMR analysis of an aliquot from the slightly hazy solution indicated complete conversion to the desired monomer, N-(1-((4-guanidinomethyl)benzyl)amino-2-methyl-1-oxopropan-2-yl)acrylamide, sodium hydrogen sulfate. ¹H-NMR (500 MHz, D₂O) d 7.17 (br s, 4H), 6.16 (m, 1H), 6.05 (m, 1H), 5.62 (m, 1H), 4.26 (s, 2H), 4.23 (s, 2H), 1.36 (s, 6H).

### Example 2. Adduct of IEM and 1-(4-(aminomethyl)benzyl)guanidine

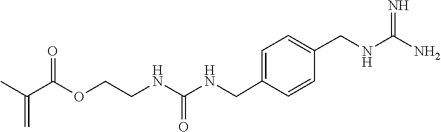 [SMILES: C=C(C)C(=O)OCCNC(=O)NCc1ccc(CNC(=N)N)cc1]

VDM reagent was replaced with IEM in the procedure of Example 1 to provide the monomer 2-(3-(4-(guanidinomethyl)benzyl)ureido)ethyl methacrylate, sodium hydrogen sulfate.

### Example 3. Adduct of IEM and 1-(3-(aminomethyl)benzyl)guanidine

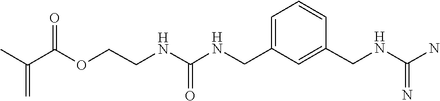

1-(4-(Aminomethyl)benzyl)guanidine sulfate was replaced with 1-(3-(aminomethyl)benzyl)guanidine sulfate in the procedure of Example 2 to provide the monomer 2-(3-(3-(guanidinomethyl)benzyl)ureido)ethyl methacrylate, sodium hydrogen sulfate.

### Example 4. Adduct of VDM and 1-(3-(aminomethyl)benzyl)guanidine

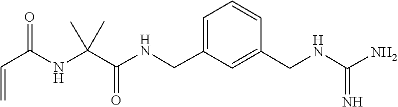 [SMILES: C=CC(=O)NC(C)(C)C(=O)NCc1cccc(CNC(=N)N)c1]

1-(4-(Aminomethyl)benzyl)guanidine sulfate was replaced with 1-(3-(aminomethyl)benzyl)guanidine sulfate in the procedure of Example 1 to provide the monomer N-(1-((3-guanidinomethyl)benzyl)amino-2-methyl-1-oxopropan-2-yl)acrylamide, sodium hydrogen sulfate.

### Example 5. Adduct of Karenz MOI-EG and 1-(4-(aminomethyl)benzyl)guanidine Sulfate

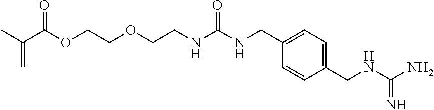 [SMILES: C=C(C)C(=O)OCCOCCNC(=O)NCc1ccc(CNC(=N)N)cc1]

VDM reagent was replaced with Karenz MOI-EG in the procedure of Example 1 to provide the monomer 2-(2-(3-(4-(guanidinomethyl)benzyl)ureido)ethoxy)ethyl methacrylate, sodium hydrogen sulfate.

### Example 6. Adduct of Karenz MOI-EG and 1-(3-(aminomethyl)benzyl)guanidine Sulfate

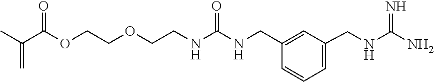 [SMILES: C=C(C)C(=O)OCCOCCNC(=O)NCc1cccc(CNC(=N)N)c1]

1-(4-(Aminomethyl)benzyl)guanidine sulfate was replaced with 1-(3-(aminomethyl)benzyl)guanidine sulfate in the procedure of Example 5 to provide the monomer, 2-(2-(3-(3-(guanidinomethyl)benzyl)ureido)ethoxy)ethyl methacrylate, sodium hydrogen sulfate.

### Comparative Example A. Adduct of IEM and Agmatine

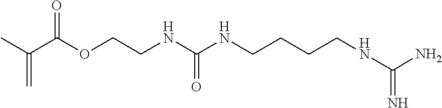 [SMILES: C=C(C)C(=O)OCCNC(=O)NCCCCNC(=N)N]

4-(2-(methacryloyloxy)ethylaminocarbonylamino)butyl guanidinium sodium sulfate (IEM-Agmatine) was prepared according to the procedure of Example 99 of U.S. Pat. No. 9,272,246 (Rasmussen).

### Comparative Example B. Adduct of IEM and 4-Aminobenzylguanidine

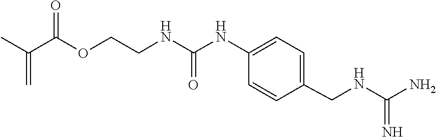 [SMILES: C=C(C)C(=O)OCCNC(=O)Nc1ccc(CNC(=N)N)cc1]

4-Aminobenzylamine (12.2 grams, 0.1 mole) was dissolved in deionized water (50 mL) in a 200 mL round bottom flask with magnetic stirring. O-Methylisourea hydrochloride (11.61 grams, 0.1 mole) dissolved in deionized water (50 mL) was added to the flask. The resulting mixture was stirred at ambient temperature (about 21° C.) for 24 hours and then cooled in an ice-water bath for 15 minutes. IEM (5.0 mL) was added to the reaction mixture by syringe. The reaction mixture was stirred for 20 minutes and then a second portion of IEM (5.0 mL) was added. The mixture was stirred for 15 minutes and then an additional portion of IEM (4.0 mL) was added by syringe. The ice-water bath was removed and the reaction mixture was stirred overnight. The precipitated product was filtered, washed with a small amount of deionized water, and dried to provide 27.4 grams of light yellow solid. ¹H-NMR analysis indicated complete conversion to the desired monomer 2-[[4-(guanidinomethyl)phenyl]carbamoylamino]ethyl prop-2-enoate hydrochloride. ¹H-NMR (500 MHz, CD₃OD) δ 1.94 (s, 3H), 3.50 (t, 2H), 4.21 (t, 2H), 4.31 (s, 2H), 5.63 (s, 1H), 6.14 (s, 1H), 7.21 (d, 2H), 7.38 (d, 2H).

### Examples 7-12 and Comparative Example 1

Nylon membranes were coated and grafted with a single monomer selected from Examples 1-6 and Comparative Example A according to the “General Procedure for Membrane Coating and UV Irradiation Grafting” described above. Coating solutions were prepared at 0.25 M monomer concentration and 0.0625% weight/weight C-BP photoinitiator concentration. The results for Ligand Density, BSA Binding Capacity, and Ligand Efficiency (molar ratio) are reported in Table 1.

TABLE 1
Results for Functionalized Membranes of
Examples 7-12 and Comparative Example 1

| | | | | Ligand |
|---|---|---|---|---|
| | | | BSA | Efficiency |
| | Monomer used | Ligand | Binding | Molar Ratio |
| | for Coating and | Density | Capacity | (ligands/ |
| Example | Grafting | (mmol/g) | (mg/mL) | protein) |
| 7 | Example 1 | 0.18 | 43 | 116 |
| 8 | Example 2 | 0.34 | 58 | 159 |
| 9 | Example 3 | 0.32 | 58 | 152 |
| 10 | Example 4 | 0.18 | 43 | 114 |
| 11 | Example 5 | 0.37 | 83 | 125 |
| 12 | Example 6 | 0.36 | 63 | 157 |
| Comparative | Comparative | 0.38 | 29 | 365 |
| Example 1 | Example A | | | |

### Examples 13-16 and Comparative Example 2

Nylon membranes were coated and grafted with a single monomer selected from Examples 1, 4 and Comparative Example A according to description of Examples 7-12 with the exception that higher concentrations of monomer were used in the coating solutions. In Example 13, the concentration of the monomer of Example 1 in the coating solution was 0.375 M and for Example 14 the concentration of the monomer of Example 1 in the coating solution was 0.5 M. In Example 15, the concentration of the monomer of Example 4 in the coating solution was 0.375 M and for Example 16 the concentration of the monomer of Example 4 in the coating solution was 0.5 M. For Comparative Example 2 the concentration of Comparative Example A monomer in the coating solution was 0.375 M. The results for Ligand Density, BSA Binding Capacity, and Ligand Efficiency (molar ratio) are reported in Table 2.

TABLE 2
Results for Functionalized Membranes of
Examples 13-16 and Comparative Example 2

| | | | | Ligand |
|---|---|---|---|---|
| | | | BSA | Efficiency |
| | Monomer used | Ligand | Binding | Molar Ratio |
| | for Coating and | Density | Capacity | (ligands/ |
| Example | Grafting | (mmol/g) | (mg/mL) | protein) |
| 13 | Example 1 | 0.22 | 61 | 98 |
| 14 | Example 1 | 0.58 | 90 | 177 |
| 15 | Example 4 | 0.30 | 67 | 123 |
| 16 | Example 4 | 0.50 | 59 | 234 |
| Comparative | Comparative | 0.57 | 60 | 264 |
| Example 2 | Example A | | | |

The results in Tables 1 and 2 show that compared to membranes grafted with IEM-Agmatine (Comparative Example A), polymer grafted membranes of Examples 7-16 have better Ligand Efficiencies when grafted to membranes at similar or lower ligand densities.

### Comparative Example 3

Nylon membranes were coated and grafted with the monomer of Comparative Example B according to the “General Procedure for Membrane Coating and UV Irradiation Grafting” described above. Coating solutions were prepared with either a 0.375 M or 0.5 M concentration of the monomer of Comparative Example B and 0.0625% weight/weight of C-BP photoinitiator. The results for Ligand Density, BSA Binding Capacity, and Ligand Efficiency (molar ratio) are reported in Table 3.

TABLE 3
Results for Functionalized Membranes of Comparative Example 3

| Concentration of | | | Ligand |
|---|---|---|---|
| Comparative Example | | | Efficiency |
| B Monomer used | Ligand | BSA Binding | Molar Ratio |
| for Coating and | Density | Capacity | (ligands/ |
| Grafting (M) | (mmol/g) | (mg/mL) | protein) |
| 0.375 | 0.51 | 4 | 3184 |
| 0.50 | 0.68 | 11 | 1641 |

The results in Table 3 show that polymer grafted membranes prepared with Comparative Example B Monomer have less BSA Binding Capacity and poorer Ligand Efficiency than the polymer grafted membranes of Examples 7-16.

### Comparative Example 4

Nylon membranes were coated and grafted with methacrylamidopropyltrimethylammonium chloride (MAPTAC) at a monomer concentration of 0.5 M according to the “General Procedure for Membrane Coating and UV Irradiation Grafting” described above. The resulting polymer grafted membrane had a Ligand Density of 0.45 mmol/g.

### Example 17. BSA Binding Capacity and Salt Tolerance

Membranes from Examples 14, 15, and Comparative Example 4 were evaluated according to the “Salt Tolerance Test Method for BSA Binding Capacity” described above. The results are reported in Table 4.

TABLE 4
Results for Functionalized Membranes of
Examples 14, 15 and Comparative Example 4

| Ionic Strength | BSA Binding Capacity (mg/mL) of Grafted Membrane | | |
|---|---|---|---|
| of Challenge | | | Comparative |
| Solution (mM) | Example 14 | Example 15 | Example 4 |
| 6 | 84 | 76 | 95 |
| 50 | 98 | 92 | 44 |
| 150 | 108 | 91 | 26 |
| 250 | 95 | 72 | 10 |

The results in Table 4 show that for grafted membranes of Examples 14 and 15, the BSA binding capacity was maintained or increased with increasing ionic strength of the challenge solution. However, for the grafted membrane of Comparative Example 4, the BSA binding capacity decreased with increasing ionic strength of the challenge solution.

1. An anion exchange separation article comprising:
a porous polymeric substrate that is a solid; and
a plurality of polymeric chains grafted to the porous polymeric substate and extending away from a surface of the porous polymeric substate, wherein the polymeric chains comprise monomeric units derived from a monomer of Formula (I) or a salt thereof 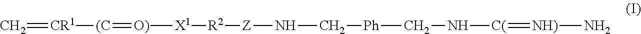
wherein
R¹ is hydrogen or methyl;
X¹ is —O— or —NH—;
R² is a (hetero)alkylene;
Z is —NH—(C═O)— or —(C═O)—; and
Ph is phenylene.

2. The anion exchange separation article of claim 1, wherein the porous polymeric substrate is a porous polymeric membrane.

3. The anion exchange separation article of claim 1, wherein the polymeric chains comprise at least 20 weight percent monomeric units derived from monomers of Formula (I) or the salt thereof.

4. The anion exchange separation article of claim 1, wherein the anion exchange separation article is salt tolerant.

5. The anion exchange separation article of claim 1, wherein the monomer of Formula (I) is one or more of the following compounds: 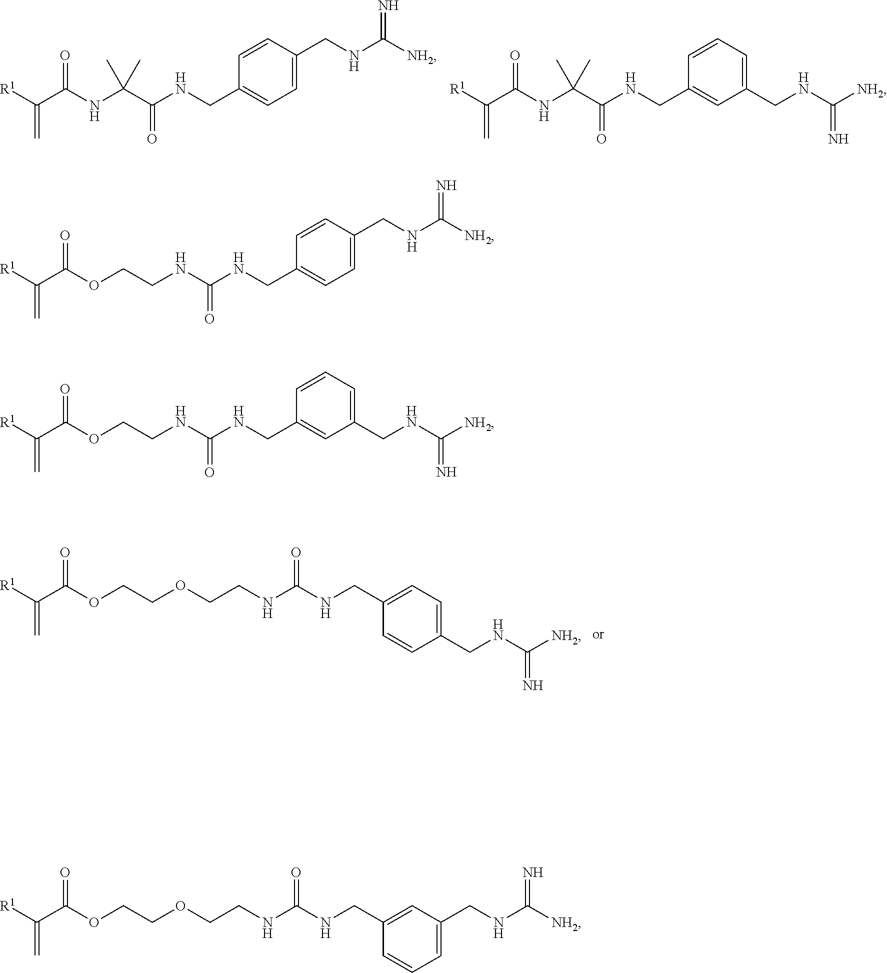
wherein R¹ is hydrogen or methyl.

6. A method of making an anion separation article, the method comprising:
providing a porous polymeric substrate that is a solid;
grafting a plurality of polymeric chains to the porous polymeric substate, wherein the polymeric chains comprise monomeric units derived from a monomer of Formula (I) 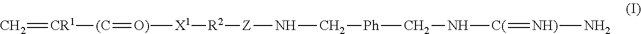
wherein
R¹ is hydrogen or methyl;
X¹ is —O— or —NH—;
R² is a (hetero)alkylene;
Z is —NH—(C═O)— or —(C═O)—; and
Ph is phenylene.

7. A method of separating a mixture of materials, the method comprising:
providing an anion exchange separation article of claim 1;
passing the mixture of materials through the anion exchange separation device, wherein the anion exchange device separates the mixture of materials based on their ionic charge.

8. The method of claim 7, wherein the anion exchange separation article is salt tolerant at ionic strength of at least 50 millimolar.

9. A monomer of Formula (I) or a salt thereof 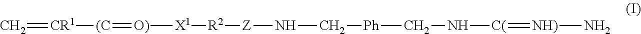
wherein
R¹ is hydrogen or methyl;
X¹ is —O— or —NH—;
R² is a (hetero)alkylene;
Z is —NH—(C═O)— or —(C═O)—; and
Ph is phenylene.

10. The monomer of claim 9, wherein the monomer of Formula (I) is 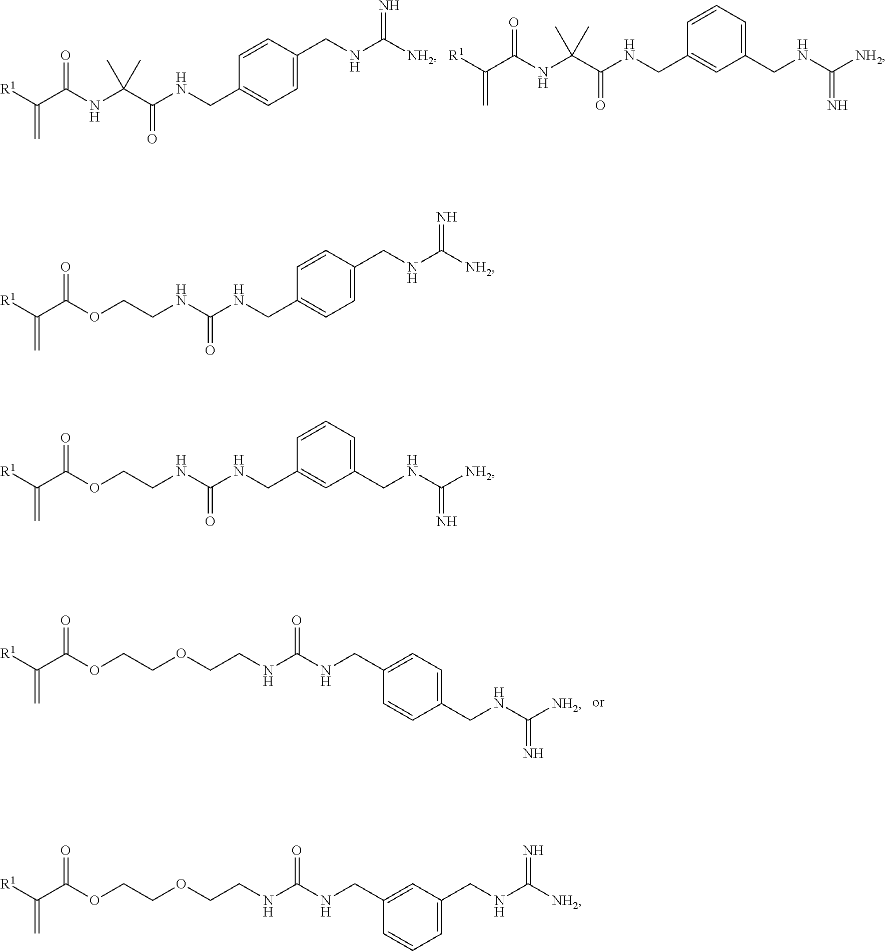
wherein R¹ is hydrogen or methyl.
